(19) United States
(12) Patent Application Publication
(10) Pub. No.: US 2026/0266791 A1
(43) Pub. Date: Sep. 10, 2026

(54) METHOD FOR DETERMINING STRENGTH OF TREE TRUNK

(71) Applicant: KING FAHD UNIVERSITY OF PETROLEUM AND MINERALS, Dhahran (SA)

(72) Inventor: Djamel OUIS, Dhahran (SA)

(73) Assignee: KING FAHD UNIVERSITY OF PETROLEUM AND MINERALS, Dhahran (SA)

(21) Appl. No.: 19/071,588

(22) Filed: Mar. 5, 2025

(51) Int. Cl. G01N 33/00; G01M 5/00

(52) U.S. Cl. CPC G01N 33/0098; G01M 5/0058

(57) ABSTRACT

A method for determining strength of a tree trunk includes connecting one end of a cable to an attachment point on the tree trunk above ground, with another end of the cable being connected through a strain gauge to a winch, wherein the winch applies a pulling force through the cable to the tree trunk until a deflection of the tree trunk meets a predefined deflection threshold. The method further includes measuring, using the strain gauge and a protector, a magnitude as well as application angle of the pulling force, respectively, and using the measured magnitude as well as application angle of the pulling force, based on a pre-established model that correlates a pulling force applied to the tree trunk with a displacement of the tree trunk, calculating a Modulus of Elasticity (MoE) of the tree trunk, that represents the strength of the tree trunk.

### BACKGROUND

### Technical Field

The present disclosure is directed to a method for determining strength of a tree trunk, and more particularly relates, to a method for determining the strength of a palm tree trunk through a pull test.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

As climate change accelerates, the frequency and intensity of wind-related disasters, such as typhoons and cyclones, are increasing. These extreme weather events cause significant damage to both natural and urban environments, particularly affecting forests and street trees. In many cases, high winds lead to the breakage and collapse of weakened, aged, or structurally compromised trees. Such incidents pose serious risks to public safety, infrastructure, and property, especially in densely populated urban areas. To mitigate these risks, it is essential to assess the structural integrity of trees and their ability to withstand wind forces. Evaluating a tree's resistance to wind pressure allows for proactive interventions, such as pruning, support installations, or removal of hazardous trees, thereby reducing potential damage and ensuring public safety. Additionally, understanding the threshold at which a tree succumbs to wind stress can aid in urban planning, forest management, and the development of strategies to enhance tree resilience in storm-prone regions.

Several techniques have been developed to assess tree strength and structural stability. Among them, digital and sensor-based systems are widely used in the field. One method, described in reference CN115299287A, involves conducting static tensile force tests by applying controlled forces to a tree from the horizontal direction and analyzing its response. This approach helps simulate wind-induced stress and provides insights into the tree's mechanical strength and breaking point. Another technique, outlined in reference CN111238944A, uses a digital micro-angle sensor attached to the tree trunk to measure angular displacement under strong winds. The collected data is processed using a predefined algorithm to determine trunk bending strain or stress levels, helping to predict potential tree failure. Similarly, reference JP2008275319A introduces a method for real-time analysis by extracting a specimen from the tree and measuring its Young's modulus, a key indicator of stiffness, using sensors such as load and displacement sensors.

Although these digital-based systems provide valuable data, they come with several limitations. The high cost of specialized sensors and equipment makes them inaccessible for widespread use, particularly in large-scale assessments. Many of these systems require intricate calibration and skilled personnel for operation, making them less practical for routine tree strength evaluations. Additionally, the equipment used in these methods is often bulky and difficult to transport, especially in remote forested areas or urban locations with restricted access. These challenges limit the efficiency and feasibility of using digital-based methods for large-scale or rapid assessments.

Given these constraints, there is a strong demand for a more practical and cost-effective alternative for assessing tree strength. An ideal system would eliminate the reliance on complex digital sensors while still providing accurate strength measurements. It should be lightweight and easy to transport, enabling use in both urban and remote forest settings. Additionally, a simpler setup that does not require specialized technical expertise would make tree assessments more accessible and efficient. Developing such a system would enhance tree risk evaluation efforts, improve disaster preparedness, and support urban and forest management strategies while ensuring public safety in regions prone to severe wind events.

### SUMMARY

In an exemplary embodiment, the present disclosure discloses a method for determining strength of a tree trunk. The method includes connecting one end of a cable to an attachment point on the tree trunk above ground, with another end of the cable being connected through a strain gauge to a winch that is fixed to an immovable structure. The method further includes operating the winch to apply a pulling force through the cable to the tree trunk, until a deflection of the tree trunk meets a predefined deflection threshold. The method further includes using the strain gauge to measure a magnitude of the pulling force. The method further includes using a protractor to measure an applying angle of the pulling force; and using the measured magnitude and the measured applying angle of the pulling force, based on a pre-established model that correlates a pulling force applied to the tree trunk with a displacement of the tree trunk, calculating a Modulus of Elasticity (MoE) of the tree trunk, as a parameter representing the strength of the tree trunk.

In an embodiment, the step of calculating the MoE of the tree trunk further includes calculating a longitudinal MoE along a fiber direction of the tree trunk, and a shearing MoE along a direction normal to the fiber direction, as the parameter representing the strength of the tree trunk.

In an embodiment, the pre-established model represents the tree trunk as a tapered solid beam that is clamped at a bottom and free at a tip.

In an embodiment, the pre-established model represents a tree canopy as a mass applied on the tapered solid beam.

In an embodiment, the pre-established model represents the tree canopy as a concentrated mass carried by the tapered solid beam.

In an embodiment, the pre-established model represents the tree canopy as a mass distributed along the tapered solid beam.

In an embodiment, the pre-established model represents the tree canopy as an ellipsoidal shape, with a top of the ellipsoidal shape extending beyond a height of the tapered solid beam.

In an embodiment, the pre-established model represents the tree canopy as a conical shape, with a top of the conical shape extending beyond a height of the tapered solid beam.

In an embodiment, the pre-established model is defined by a plurality of parameters, and values of the plurality of parameters are determined based on geometry and physical properties of the tree trunk and the tree canopy, and the geometry and physical properties include a length, a cross-section, a tapering of the tree trunk, and a foliage mass of the tree canopy.

In an embodiment, the pre-established model represents the tree trunk as a number (N) of tree trunk elements, wherein a displacement and a rotation of a i-th tree trunk element are determined based on a shear force and a moment acting at the i-th tree trunk element and a displacement and a rotation of an (i−1)-th tree trunk element.

In an embodiment, the pulling force is applied through the cable to the tree trunk, until the attachment point deflects from a rest position thereof to a predefined distance threshold in a horizontal direction.

In an embodiment, the pulling force is applied through the cable to the tree trunk, until a tip of the tree trunk deflects from a rest position thereof to a predefined distance threshold in a horizontal direction.

In an embodiment, the protractor is used to measure an angle of the cable with respect to a horizontal direction.

In an embodiment, the protractor is used to measure an angle of the cable with respect to a vertical direction.

In an embodiment, the tree trunk has a length of 12 meters, and the pulling force is applied at an applying angle of 45°, until the deflection of the tree trunk reaches 0.5 meter.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an exemplary illustration of an environment that includes a setup for determining strength of a tree trunk, according to certain embodiments of the present disclosure.

FIG. 2 illustrates a mathematical model of a tree trunk under application of a pull force, according to certain embodiments.

FIG. 3A illustrates a canopy model of a tree trunk as an ellipsoidal shape of a foliage above the tree trunk, according to certain embodiments.

FIG. 3B illustrates a canopy model 310 of the tree trunk as one or more truncated cones by approximating an ellipsoidal shape of a canopy, according to certain embodiments.

FIG. 4A illustrates beam bending of a general beam under application of a force, according to certain embodiments.

FIG. 4B illustrates another representation of beam bending of a general beam under application of a force, according to certain embodiments.

FIG. 5 illustrates an exemplary environment for computation of a longitudinal MoE (E) and a shearing MoE (G) for a tree trunk, according to an exemplary embodiment.

FIG. 6 illustrates a flowchart of a method for determining strength of a tree trunk, according to certain embodiments.

FIG. 7 is an illustration of a non-limiting example of details of computing hardware used in the computing system, according to certain embodiments.

FIG. 8 is an exemplary schematic diagram of a data processing system used within the computing system, according to certain embodiments.

FIG. 9 is an exemplary schematic diagram of a processor used with the computing system, according to certain embodiments.

FIG. 10 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise. The drawings are generally drawn to scale unless specified otherwise or illustrating schematic structures or flowcharts.

Furthermore, the terms “approximately,” “approximate,” “about,” and similar terms generally refer to ranges that include the identified value within a margin of 20%, 10%, or preferably 5%, and any values therebetween.

Furthermore, the terms tree, tree trunk, winched tree, etc., are used as synonyms throughout the disclosure and used interchangeably.

Aspects of this disclosure are directed to a method for determining the strength of a tree trunk through a pull test. The pull test involves applying force to the tree trunk using a suitable means. Before performing the method, one end of a cable is connected to an attachment point on the trunk above the ground, while the other end is connected to a winch via a strain gauge. The winch is fixed to an immovable structure. The winch is operated to extend the cable, thereby pulling the trunk until the attachment point or the top of the trunk reaches a predefined horizontal displacement from its rest position. By measuring the pulling force with the strain gauge and measuring the angle of the cable relative to the horizontal or vertical plane, the Modulus of Elasticity (MoE) of the trunk material can be calculated using a model that correlates the pulling force with the trunk displacement. Compared to the prior art, the method disclosed in the present invention uses a strain gauge and a protractor instead of sensors or digital equipment, providing a faster and more affordable test. Moreover, the tree trunk considered in the present invention is a palm tree. However, the method is not restrictive and can equally be applied to any type of tree to determine its trunk strength. A detailed aspect of the invention is provided in the following description.

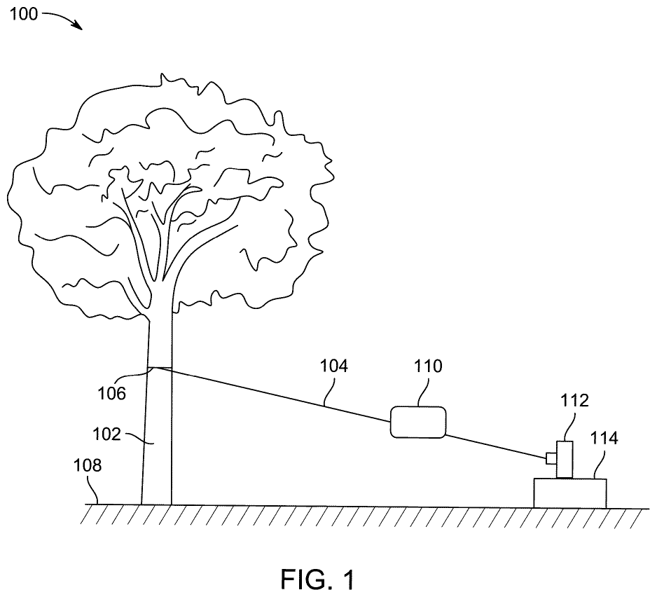

FIG. 1 is an exemplary illustration of an environment 100 that includes a setup for determining the strength of a tree trunk 102, according to certain embodiments of the present disclosure. The setup includes a cable 104, a strain gauge 110, and a mechanical winch 112 fixed to an immovable structure 114. The cable 104 may refer to a rope or a metallic cable. Furthermore, the strain gauge 110 may be selected from a list that includes a piezoelectric load cell, a hydraulic load cell, a capacitive load cell, or similar devices known in the art. The winch 112 may be a manually operated winch, an automatically operated winch, or a command-based winch. Moreover, the winch 112 may be electrically or hydraulic operated winch. The immovable structure 114 may refer to an anchoring tree to which the winch 112 is tied. In another embodiment, the immovable structure 114 refers to a heavy vehicle on which the winch 112 is immovably installed. The setup additionally includes a protractor (not shown) to be used simultaneously while determining the strength of the tree trunk 102.

The environment 100 shows a tree 102, also referred to as the tree trunk 102 or the winched tree 102, whose strength is to be determined by the pull test using the described setup. One end of the cable 104 is manually tied to a defined attachment point 106 on the tree trunk 102. The attachment point 106 is located at a certain height above the surface of the ground 108. The other end of the cable 104 is connected to the winch 112. The cable 104 connects the winch 112 and the tree trunk 102 through the strain gauge 110. In other words, the strain gauge 110 is connected to the cable 104 somewhere between the attachment point 106 and the winch 112. In an embodiment, the positions of the strain gauge 110 and the winch 112 may be interchanged, such that one end of the winch 112 is connected directly to the tree trunk 102 via the cable 104, while the strain gauge 110 is installed on the immovable structure 114 and connected to the other end of the winch 112.

Once the setup is connected as described and illustrated with reference to FIG. 1, the winch 112 is operated to determine the strength of the tree trunk 102. Operating the winch 112 causes the exertion of a pull force through the cable 104 to the tree trunk 102. The winch 112 is operated until the deflection of the tree trunk 102 meets a predefined deflection threshold in the horizontal direction. In an embodiment, the pulling force is applied through the cable 104 to the tree trunk 102, until either the attachment point 106 or the tip of the tree trunk 102 deflects from its rest position to a predefined distance threshold in the horizontal direction. For example, upon the application of the pull force, the entire structure of the tree, including the attachment point 106 as well as the tip of the tree trunk 102, begins to lean toward the pulling direction with a small displacement in the horizontal direction, while simultaneously providing a resistance force against the applied force. In this case, the horizontal distance traveled by the tip of the tree trunk 102 is greater than the horizontal distance traveled by the attachment point 106 of the tree trunk 102.

Once the tree trunk 102, either the tip of the tree trunk 102 or the attachment point 106, is deflected to the minimum predefined threshold distance, the strain gauge 110 begins to display the magnitude of the pulling force currently acting on the tree trunk 102 at the attachment point 106. The magnitude of the pulling force displayed on the strain gauge 110 is measured either manually or automatically. Moreover, before the application of the pulling force, the cable 104 forms a specific angle with respect to the ground 108. Upon the application of the pulling force, the angle subtended by the cable 104 at the attachment point 106 changes as the tree trunk 102 leans slightly toward the direction of the applied force. Therefore, when measuring the magnitude of the pulling force, the protractor is used to manually measure the angle of the pulling force on the cable 104 with respect to the horizontal direction or the ground 108. In another embodiment, the protractor may be used to manually measure the angle of the pulling force on the cable 104 with respect to the vertical direction or the fiber direction of the tree trunk 102. The angle may be measured in degrees or radians. In one embodiment, the winch 112 may be integrated with the protractor to manually measure the angle with respect to the ground 108 at the time of application of the pulling force. In another embodiment, the protractor may be configured for automatic use when applying the pulling force. For example, an automatic system (not shown) integrated with the winch 112 may automatically position the protractor to measure the angle of force application on the cable 104 as soon as the attachment point 106 or the tip of the tree trunk 102 deflects from its resting position to the predefined threshold distance in the horizontal direction. In another embodiment, a computer system (not shown), such as a laptop, desktop, a mobile or alike, may be electrically coupled with the strain gauge 110, winch 112, and protractor to automatically read the magnitude of the pulling force and measure the angle of force application with respect to the ground 108 by automatically using the protector to measure the angle, as soon as the attachment point 106 or the tip of the tree trunk 102 deflects from its resting position to the predefined threshold distance in the horizontal direction. The computer system described herein may refer to a fully automatic system wherein the computer system may be electrically coupled with the strain gauge 110, winch 112, and the protractor. The computer system, under the control of an user input or control, commands to operate the winch 112 to apply a pulling force to the cable 104, commands to read the magnitude of the pulling force on the strain gauge 110 and measure the angle of application of the force with respect to the ground 108 as soon as the attachment point 106 or the tip of the tree trunk 102 deflects from its resting position to the predefined threshold distance in the horizontal direction.

Once the magnitude of the pulling force as well as the angle of the pulling force are measured, a pre-established model is used to automatically compute the strength of the tree trunk 102. The pre-established model consists of a set of mathematical equations used to compute the Modulus of Elasticity (MoE) of the tree trunk 102. The pre-calibrated model includes a plurality of unknown quantities, as shown in equations 1 and 2 below,

$${y}_{i} = {y}_{i - 1} + {θ}_{i - 1} Δ x + {Q}_{i} \frac{Δ x}{G {A}_{s i}} + {M}_{i} \frac{Δ {x}^{2}}{2 {EI}_{i}} + {Q}_{i} \frac{Δ {x}^{3}}{3 {EI}_{i}} \qquad ( 1 )$$

$${θ}_{i} = {θ}_{i - 1} + {Q}_{i} \frac{1}{G {A}_{s i}} + {M}_{i} \frac{Δ x}{{EI}_{i}} + {Q}_{i} \frac{Δ {x}^{2}}{2 {EI}_{i}} \qquad ( 2 )$$

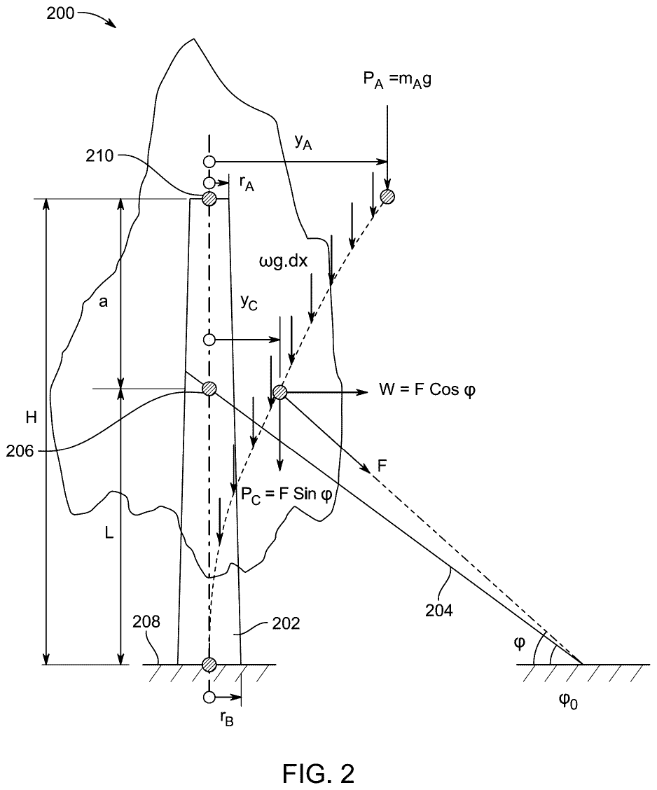

The pre-established model is defined by a plurality of parameters described in equation 1 and 2. The value of each parameter is based on the geometry and physical properties of the tree trunk 102 and the tree canopy. The tree canopy and their property are described later in the description. Moreover, the geometry and physical properties include one or more parameters such as a length and a cross-section area of the tree trunk 102, a tapering of the tree trunk 102, and a foliage mass of the tree canopy. The detailed explanation of mathematical equations 1 and 2 along with plurality of parameters are provided in FIG. 2-FIG. 4, which are described later in the description. The pre-established model shows a relationship between the pulling force (Qᵢ) applied to the tree trunk 102 and the displacement (yᵢ) of the tree trunk 102. The term ‘i’ indicates the location of an i^{th} node or element from the bottom of the tree trunk 102. Equations 1 and 2 correlate the two parameters, i.e., an applied pulling force (Qᵢ) and the displacement (yᵢ) of the tree trunk 102, to calculate the MoE of the tree trunk 102. The value of the MoE represents the strength of the tree trunk 102. In order to compute the MoE, the pre-established model initially computes various other parameters of the tree trunk 102, such as the mass of the foliage area (mF), second moment of area (Iᵢ), bending moment (Mᵢ), shear area (Aₛᵢ), rotation angle (θᵢ), and density (ρ), which are described in detail in FIGS. 2-FIG. 4. In an embodiment, the pre-established model is preloaded in a memory (not shown) of the computer system (not shown) described earlier and the pre-established model is executed, using one or more processors of the computer system (not shown).

FIG. 2 illustrates a mathematical model 200 of a tree trunk 202 under the application of a pull force F, according to an embodiment. The tree trunk 202 is representative of the tree trunk 102 in FIG. 1. The tree trunk 202 has a height H from the surface of the ground 208. The model represents the tree trunk 202 as a tapered solid beam or a cantilever beam that has two ends. One end of the tapered solid beam or cantilever beam is fixed or clamped to the surface of the ground 208. The clamped bottom of the tree trunk 202 has a radius r_{B}. The other end, known as the tip 210 of the tree trunk 202, is free to move and has a radius r_{A}. Since the solid beam is assumed to have uniform tapering, the radius r_{B} is greater than the radius r_{A}.

A point 206 on the tree trunk 202 indicates the location where a cable 204, which is representative of the cable 104 in FIG. 1, is tied. The point 206 is at a distance ‘L’ from the surface of the ground 208 or at a distance ‘a’ from the tip 210 of the tree trunk 202. The other end of the cable 204 is attached to the winch (not shown), as shown in FIG. 1. Although the winch and the strain gauge are not shown in FIG. 2, it is customary to assume their presence in accordance with the invention. Before the application of the pull force F, the cable 204 subtends a specific angle φ₀ with respect to the ground surface 208. A pull force F is applied at point 206 through the cable 204. Upon application of the pulling force F, the tree trunk 202 bends forward, causing point 206 to lean forward by a specific distance y_{c}. In other words, point 206 moves forward by a distance y_{c}, whereas the tip 210 moves forward by a distance y_{A}. Since the tip 210 is located toward the free end of the tree trunk 102, the distance y_{A} are greater than the distance y_{c}. Additionally, the application of the pulling force changes the initial angle of the pulling force F from φ₀ to φ. This is the angle at which the pulling force F is applied to the cable 204.

The pulling force F can be resolved into horizontal and vertical (downward) components.

The force F in the horizontal direction is called the shearing force and is represented by W, which acts along a direction normal to the fiber direction, whereas the force F in the vertically downward direction is called the longitudinal force and is represented by P_{c}, which acts along the fiber direction of the tree trunk 202. The fiber direction refers to the direction that runs along the central axis of the tree trunk 202. Therefore, the shearing force acts in a direction normal to the fiber direction. Mathematically, if the pulling force F is applied at an angle φ with respect to the ground surface 208, the shearing force W and the longitudinal force P_{c} can be expressed as follows:

$$W = F Cos ( φ ) \qquad ( 3 )$$

$${P}_{c} = F Sin ( φ ) \qquad ( 4 )$$

The shear force W is applied equally to all points on the tree trunk 202 that are below the point 206 where the force F is applied. Above the point 206, the shear force W becomes zero, and only the longitudinal force P_{c} applies.

Accordingly, two forces act simultaneously at point 206. This is the case considered for only one point 206. Similarly, the tree trunk 202 is assumed to be made up of a plurality of uniformly spaced elements located between the bottom of the tree trunk 202 and the tip point 210. The pulling force Facts on each of these elements. However, from the bottom of the tree trunk 202 to the point of application of the pulling force F, both components of the pulling force, i.e., the horizontal component W and the vertically downward component P_{c} act. Above the point 206 of the application of the pulling force F, only the vertically downward component P_{c} acts. Additionally, only one vertically downward component of the force, with a magnitude of m_{A}*g, acts at the tip 210. Moreover, it is assumed that the inclined force F is applied by the cable 204 with a fixed anchor point, i.e., using the winch 112 in FIG. 1. Therefore, the inclination angle φ increases slightly as the tree trunk 202 deflects. Accordingly, φ can also be represented in terms of the initial angle φ₀ and the distance of the point of application from the ground surface L as follows

$$Φ = {Tan}^{- 1} [ L / ( ( L / Tan {φ}_{0} ) - {y}_{c} ) ] \qquad ( 5 )$$

where y_{c}=deflection at the force of application point 206, and φ₀=initial inclination angle when the tree trunk 202 is undeflected.

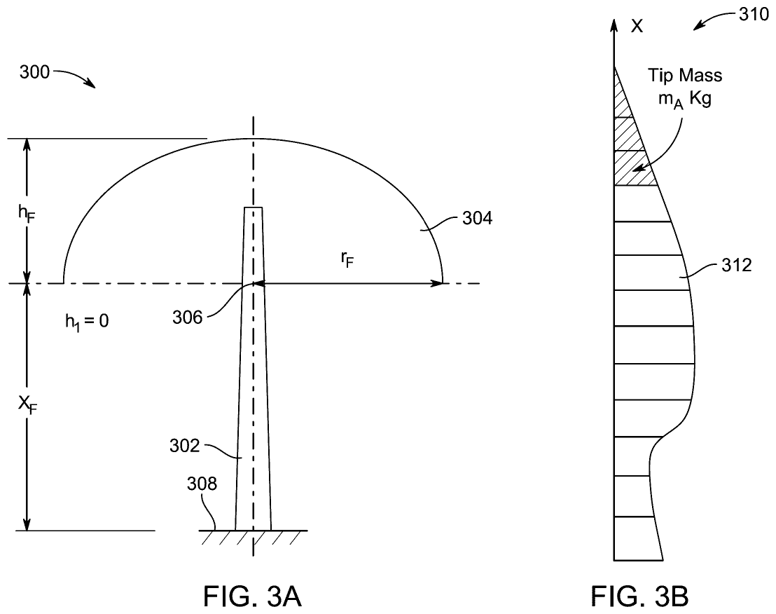

Other parameters, such as the mass of the foliage (mf) above the tree trunk 202, are also necessary to compute in order to determine the modulus of elasticity of the tree trunk 202, since the foliage part of the tree trunk 202 imparts a significant amount of mass on the tree trunk 202. This mathematics is illustrated in FIGS. 3A and 3B in detail.

FIG. 3A illustrates a canopy model 300 of a tree trunk 302 as an ellipsoidal shape of the foliage above the tree trunk 302, according to an embodiment. The canopy model 300 assumes that the whole tree consists of a solid mass of the tree trunk 302 along with a certain mass of foliage, which can be thought of as a canopy 304 above the tree trunk 302. Accordingly, the canopy model 300 treats the foliage as a tree canopy 304, which is referred to as a mass applied on the tapered solid beam 302. As such, the pre-established model represents the whole tree as the mass of foliage, with the canopy 304 positioned on the tapered solid beam 302. The canopy 304 can be considered to have the shape of an ellipse. This ellipsoidal shape extends beyond the height (x_{F}) of the tapered solid beam 302, and the canopy 304, having an ellipsoidal shape, is assumed to have a concentrated mass carried by the tapered solid beam 302. The tree trunk 302 may be representative of the tree trunks 102 and 202 in FIG. 1 and FIG. 2, respectively. The total height of the tree trunk 302 is represented as x_{F}+h_{F}, where x_{F} indicates the height of the tree trunk 302 from the ground surface 308 immediately below the canopy 304, and h_{F} indicates the height of the canopy 304 above the tree trunk 302. Point 306 refers to the point at which the ellipsoidal shape of the canopy 304 has a radius r_{F} that extends outward with respect to the solid beam 302.

For the ellipsoidal canopy geometrical model, a user can specify the maximum canopy radius r_{F}, the height (x_{F}) above the ground surface 308 where the canopy 304 starts, the height above the canopy base to the maximum radius h₁, and the height of the canopy h_{F}. To model a semi-ellipsoidal shape with the maximum radius r_{F} at the base of the canopy 304, h₁ is set to zero. The ellipsoidal model of the foliage allows for a ‘tall’ deciduous tree canopy where h_{F}>2r_{F}, or a wider, flatter canopy, such as a palm tree, where h_{F} is less than 2r_{F}. When h_{F}=2r_{F} and h₁=r_{F}, the canopy 304 may be considered as a sphere. When h_{F}=r_{F} and h₁=0, the canopy 304 may be considered as a flat-bottomed hemisphere.

The radius of the canopy r_{c} at position x above the ground surface 308 is given as:

$${r}_{c} ( x ) = {r}_{F} \sqrt{1 - \frac{{( x - {x}_{F} - {h}_{1} )}^{2}}{{( {h}_{F} - {h}_{1} )}^{2}}} \qquad ( 6 )$$

Since, the whole tree is assumed to be made up of small elemental elements, the radius of the canopy 304 at the i^{th} node (i.e. xᵢ≥x_{F}) can be given as below:

$${r}_{c i} = {r}_{F} \sqrt{1 - \frac{{( {x}_{i} - {x}_{F} - {h}_{1} )}^{2}}{{( {h}_{F} - {h}_{1} )}^{2}}} \qquad ( 7 )$$

The total volume (V_{F}) of the foliage canopy 304 is given as:

$${V}_{F} = \frac{π}{3} {r}_{F}^{2} \frac{{h}_{F}^{2}}{{( {h}_{F} - {h}_{1} )}^{2}} ( 2 {h}_{F} - 3 {h}_{1} ) \qquad ( 8 )$$

Accordingly, the effective density of the foliage canopy 304 can be given as below:

$${ρ}_{F} = \frac{{m}_{F}}{{V}_{F}} = \frac{3 {{m}_{F} ( {h}_{F} - {h}_{1} )}^{2}}{π {r}_{F}^{2} {h}_{F}^{2} ( 2 {h}_{F} - 3 {h}_{1} )} \qquad ( 9 )$$

FIG. 3B illustrates a canopy model 310 of the tree trunk as one or more truncated cones 312, which approximate the ellipsoidal shape of the canopy, according to an embodiment. In this case, the pre-established model also considers the tree canopy as a mass of foliage that is distributed along the tapered solid beam 302 throughout its length, with the ellipsoidal shape being truncated into cones connected one after another. As such, the tree canopy 304 may be assumed to have a conical shape, with the top of the conical shape extending beyond the height of the tapered solid beam 302. Here, the tip mass may be considered to be located at the top of the truncated cone. Additionally, the conical shape model assumes that the entire mass of the tree, including the mass of the foliage and the mass of the tree trunk, is distributed along the tree trunk per unit length in the form of truncated cones 312. When the foliage mass is apportioned to the nodes or elements within the canopy by approximating the ellipsoidal slices as truncated cones, a small error is introduced when the overall summed node masses are compared to the total foliage mass specified by the user.

Where the top of the canopy extends beyond the height of the beam, i.e., x_{F}+h_{F}>H, the mass of foliage 304 in the uppermost part is computed as partial ellipsoidal slice and lumped at the tip node. The mass of the foliage (m_{FA}) in the uppermost part is computed using the equation below:

$${m}_{F A} = \frac{π}{3} {ρ}_{F} {r}_{F}^{2} \frac{{( {h}_{F} + {x}_{F} - H + Δ {x}_{A} / 2 )}^{2}}{{( {h}_{F} - {h}_{1} )}^{2}} ( 2 {h}_{F} - 3 {h}_{1} - {x}_{F} + H - Δ {x}_{A} / 2 ) \qquad ( 10 )$$

where effective density (ρ) of the foliage canopy 304 is used from equation 9.

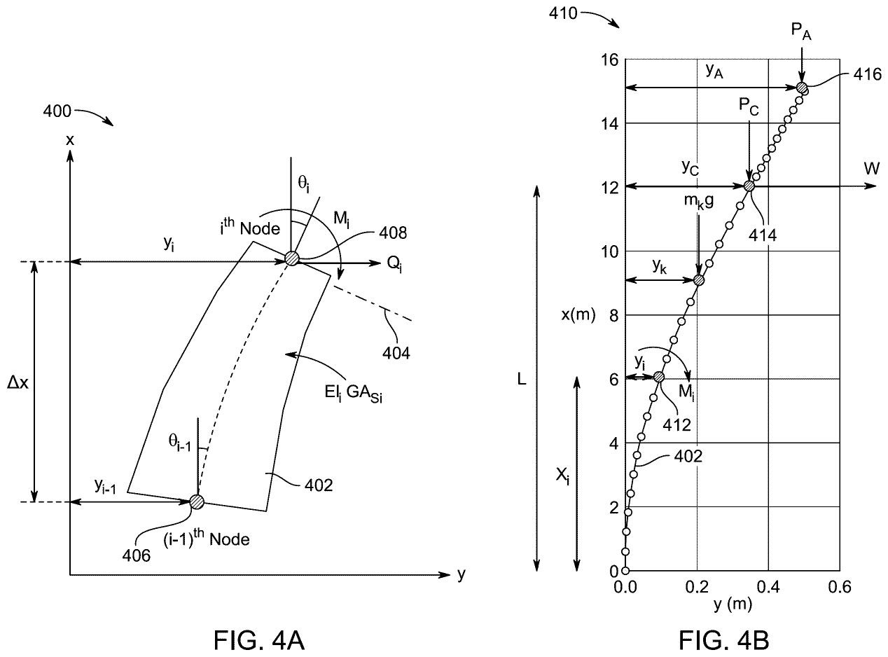

Considering the plurality of equations from 6 to 10, the mass of the foliage (m_{FA}) of the tree trunk 302 can be computed while calculating the modulus of elasticity of the tree trunk 302 to determine its strength. Since equations 1 and 2 include a plurality of parameters to be computed before determining the values of modulus of elasticity (E) and (G), their calculation is described in detail in FIG. 4A and FIG. 4B, considering the bending moment Mᵢ of a general beam.

FIG. 4A illustrates beam bending 400 of a general beam 402 under application of a force F, according to an embodiment. The beam 402 is analogous to a tree trunk 102, 202, and 302 in FIG. 1, FIG. 2, and FIG. 3, respectively. The beam 402 is represented over X and Y axis to illustrates its movement. Dashed line 404 is analogous to a cable for providing a pulling force F to the beam 402 at, for example, a point 408, which causes a shear force Qᵢ to act on an element 408 in the horizontal direction. Application of the shear force Qᵢ causes a force on every node or element of the beam 402, and accordingly every element of the beam 402 bends towards the application of the shear force Qᵢ. For example, node i bends by the angle θᵢ from the normal surface of the shear area of that node. Similarly, node i−1 bends by the angle θᵢ₋₁ from the normal surface of the shear area of that node. A coordinate yᵢ represents the location of the i^{th} node on the Y axis at the point 408, which corresponds to the shearing movement of the point 406 on the Y axis due to the application of the shear force Qᵢ at the point 408. Corresponding coordinate xᵢ represents the location of the i^{th} node on the X axis. Similarly, a coordinate yᵢ₋₁ represents the location of the (i−1)^{th} node on the Y axis at point 406, which corresponds to the shearing movement of the point 406 on the Y axis due to application of the shear force Qᵢ at the point 408. Corresponding coordinate xᵢ₋₁ represents the location of the (i−1)th node on the X axis. Application of the shearing force Qᵢ causes an incremental length Δx unit between i^{th} node and (i−1)^{th} node.

The displacement yᵢ and the rotation θᵢ of the i^{th} node are found from the applied shear force Qᵢ and the moment Mᵢ acting at the node i and the displacement yᵢ₋₁ and the rotation θᵢ₋₁ of the node (i−1)^{th} as described in Equation 1 and 2 earlier. As such, the pre-established model represents the tree trunk as a number of tree trunk elements or nodes, and a displacement yᵢ and a rotation θᵢ of a i^{th} tree trunk element or node are determined based on a shear force Qi and a moment (Mi) acting at the i^{th} tree trunk element or node and a displacement yᵢ₋₁ and a rotation θᵢ₋₁ of (i−1)^{th} tree trunk element or node.

Mathematically the equation 1 and 2 describing the plurality of parameters are again given below:

$${y}_{i} = {y}_{i - 1} + {θ}_{i - 1} Δ x + {Q}_{i} \frac{Δ x}{G {A}_{s i}} + {M}_{i} \frac{Δ {x}^{2}}{2 {EI}_{i}} + {Q}_{i} \frac{Δ {x}^{3}}{3 {EI}_{i}} \qquad ( 1 )$$

$${θ}_{i} = {θ}_{i - 1} + {Q}_{i} \frac{1}{G {A}_{s i}} + {M}_{i} \frac{Δ x}{{EI}_{i}} + {Q}_{i} \frac{Δ {x}^{2}}{2 {EI}_{i}} \qquad ( 2 )$$

where, Δx=Incremental length of the beam element between the (i−1)^{th} and i^{th} node,
Ii=Second moment of area of i^{th} node,
A_{Si}=Effective shear area of the node,
EIᵢ=Flexural stiffness of the element,
GAₛᵢ=Shear stiffness of the element,
E=Flexural modulus and is assumed as constant throughout the length of the beam, and
G=Shear modulus and is assumed as constant throughout the length of the beam.

The second moment of area Iᵢ and the effective shear area Aₛᵢ vary with position of the node. Moreover, both are taken as arithmetic mean for the cross-sections at the upper and lower nodes. Mathematically, for a tapering solid circular cross-section area of a beam, their calculation formula is given in equation 10 and 11 respectively, as below:

$${I}_{i} = \frac{1}{2} ( \frac{π}{4} {r}_{i}^{4} + \frac{π}{4} {r}_{i - 1}^{4} ) = \frac{π}{8} ( {r}_{i}^{4} + {r}_{i - 1}^{4} ) \qquad ( 11 )$$

$${A}_{s i} = \frac{1}{2} ( \frac{9 π}{10} {r}_{i}^{2} + \frac{9 π}{10} {r}_{i - 1}^{2} ) = \frac{9 π}{20} ( {r}_{i}^{2} + {r}_{i - 1}^{2} ) \qquad ( 12 )$$

Moreover, for linearly tapering solid trunk, the radius at i^{th} node can be given by equation 13, as below:

$${r}_{i} = {r}_{B} - \frac{( {x}_{B} - {x}_{A} )}{H} {x}_{i} \qquad ( 13 )$$

where,
Xᵢ is the vertical coordinate of the i^{th} node that is measured upward from the base.

When the shear force Qᵢ is applied at i^{th} node,

$${Q}_{i} = W for 0 \leq {x}_{i} \leq L \qquad ( 14 )$$

$${Q}_{i} = 0 for L < {x}_{i} \leq H \qquad ( 15 )$$

As such, the shear force Qi at all nodes that lie above the applied force F is 0.

FIG. 4B illustrates another representation of beam bending 410 of a general beam 402 under the application of a force, according to an embodiment. Upon application of the force F at a point 414, the beam 402, that is representative of the tree trunk 102, 202, 302 in FIGS. 1, 2, and 3, each point of the node, such as nodes 412, 414, and 416, etc., experience a shearing force. For example, at the point 414 of application of the force F, the shearing force Q (=W) acts in horizontal direction. yᵢ, yₖ, and y_{A} illustrate displacements of the corresponding node in the horizontal direction. Further, the axial component at a plurality of nodes can be represented by the mathematical equation below:

$${P}_{c} = F sin ( θ ) = Axial load component acting at the point of application of the force F \qquad ( 16 )$$

$${P}_{A} = {m}_{A} * g = Axial load component acting at the tip of the beam 402 \qquad ( 17 )$$

where m_{A}=Concentrated mass at the tip of the beam 402, and
g=Acceleration due to gravity=9.81 ms⁻².

Moreover, the bending moment Mᵢ at the i^{th} node depends on both the lateral component of the force F and the axial forces and their deflected lateral offsets acting above the node. The bending Mᵢ tends to rotate the corresponding node at i^{th} location. Mathematically,

$${M}_{i} = W ( L - {x}_{i} ) + {P}_{c} ( {y}_{c} - {y}_{i} ) + {P}_{A} ( {y}_{A} - {y}_{i} ) + {M}_{m i} for 0 \leq {x}_{i} \leq L ; and \qquad ( 18 )$$

$${M}_{i} = {P}_{A} ( {y}_{A} - {y}_{i} ) + {M}_{m i} for L < {x}_{i} \leq H \qquad ( 19 )$$

where yᵢ is the deflection at the i^{th} node and yₐ is the tip point deflection.

Mₘᵢ=Moment due to weight of the distributed mass (i.e., foliage and the tree trunk) above the node, Mathematically, Mₘᵢ can be given as below:

$${M}_{m i} = {\sum }_{k = i + 1}^{N} {m}_{k} g ( {y}_{k} - {y}_{i} ) \qquad ( 20 )$$

where,
Mₖ=Lumped masses,
yₖ=Lateral deflections at k^{th} node above the i^{th} node, and
N=Total number of nodes.

Equation 20 can also be re-written as below:

$${M}_{m i} = g ( {\sum }_{k = i + 1}^{N} {m}_{k} {y}_{k} - {y}_{i} {\sum }_{k = i + 1}^{N} {m}_{k} ) \qquad ( 21 )$$

Now considering the plurality of equations provided with reference to FIGS. 2-4, referring back to FIG. 1 for computing the modulus of elasticity (E and G) of the tree trunk 102 using equation 1 and 2, the equation 1 can be approximated as below:

$${y}_{A} = β \frac{W {L}^{3}}{3 {EI}_{A}} \qquad ( 22 )$$

where y_{A}=Tip deflection upon application of the force,
β=The ratio of the thickness of the tree trunk at the base to that at the tip,
W=Shear component the applied force F,
L=Length of the tree trunk at which the force is applied,
E=longitudinal MoE along a fiber direction of the tree trunk 102, and
I_{A}=second moment of area of the tree trunk 102.

Based upon readings on the strain gauge 110, the force value (F) applied on the cable 104 is identified. At the same time, the angle φ of the application angle of the force is identified by the protector. When the Force F is identified, shearing force W is identified by using equation 3 described earlier.

$$W = F Cos ( φ ) \qquad ( 3 )$$

The equation 3 provides the value of the shearing force W applied horizontally at the attachment point 106 of application of the force F.

The Value L is identified by measuring the height of the tree trunk 102 from the ground 108 till the attachment point 106 of application of the force F. This value is measured manually.

The value of y_{A} is identified by measuring the lateral deflection of the tip of the tree trunk 102 upon application of the force F. This value is measured manually.

The value of β is identified by measuring the ratio of the thickness of the tree trunk at the base (2*rb) to that at the tip (2*rag). This value is measured manually.

The value of second moment of area I_{A} of the tree trunk 102 is computed by using equation 11 as described earlier.

$${I}_{i} = \frac{1}{2} ( \frac{π}{4} {r}_{i}^{4} + \frac{π}{4} {r}_{i - 1}^{4} ) = \frac{π}{8} ( {r}_{i}^{4} + {r}_{i - 1}^{4} ) \qquad ( 11 )$$

Here, i=A (tip of the tree trunk).

The value of rᵢ equation 11 is computed using equation 13 as below:

$${r}_{i} = {r}_{B} - \frac{( {x}_{B} - {x}_{A} )}{H} {x}_{i} \qquad ( 13 )$$

Putting the value of rᵢ back into equation 11 yields the value of I_{A}.

Now rearranging equation 22 for computing E as below,

$$E = β \cdot W \cdot {L}^{3} / {y}_{A} \cdot {I}_{A} \qquad ( 23 )$$

The value of E provides a longitudinal or flexural MoE (E) along a fiber direction of the tree trunk 102.

Now, in order to identify the shearing MoE (G) along a direction normal to the fiber direction, equation 2 is used and the computed value of E using equation 23 is used in equation 2, as below

$${θ}_{i} = {θ}_{i - 1} + {Q}_{i} \frac{1}{G {A}_{s i}} + {M}_{i} \frac{Δ x}{{EI}_{i}} + {Q}_{i} \frac{Δ {x}^{2}}{2 {EI}_{i}}$$

Here, Qᵢ=W=F Cos (φ) is already computed using equation 3.
The value of A_{Si} is computed using equation 12, as described earlier and below:

$${A}_{si} = \frac{1}{2} ( \frac{9 π}{10} {r}_{i}^{2} + \frac{9 π}{10} {r}_{i - 1}^{2} ) = \frac{9 π}{10} {r}_{i}^{2} ( {r}_{i}^{2} + {r}_{i - 1}^{2} ) ; \qquad ( 12 )$$

And using equation 18 and 19 to compute the value of Mᵢ, as described earlier and below:

$${M}_{i} = W ( L - {x}_{i} ) + {P}_{c} ( {y}_{c} - {y}_{i} ) + {P}_{A} ( {y}_{A} - {y}_{i} ) + {M}_{m i} for 0 \leq {x}_{i} \leq L ; and \qquad ( 18 )$$

$${M}_{i} = {P}_{A} ( {y}_{A} - {y}_{i} ) + {M}_{m i} for L < {x}_{i} \leq H \qquad ( 19 )$$

The pre-established model also computes the value of θᵢ and θᵢ₋₁ and Δx.

When all such values such as θᵢ, θᵢ₋₁, Qᵢ, Aₛᵢ, Δx, E and Iᵢ are computed, equation 2 is rearranged to compute the value of G (shearing MoE) along a direction normal to the fiber direction.

As such, the value of the longitudinal or flexural MoE along the fiber direction of the tree trunk 102 and the shearing MoE along a direction normal to the fiber direction are computed using the pre-established model. The two parameters, i.e., the longitudinal or flexural MoE and the shearing MoE, collectively indicate the parameter that determines the strength of the tree trunk 102. The two parameters are collectively referred to as the modulus of elasticity (MoE). While experimentally performing the computation of the modulus of elasticity, plurality of parameters, such as a predetermined deflection of the lateral tip of 0.5 meter for a palm tree of height 12 meters when applied with a force at an application angle of 45° from the ground surface 108, it was found that a force of around 5700N was necessary for such tip deflection of the palm tree.

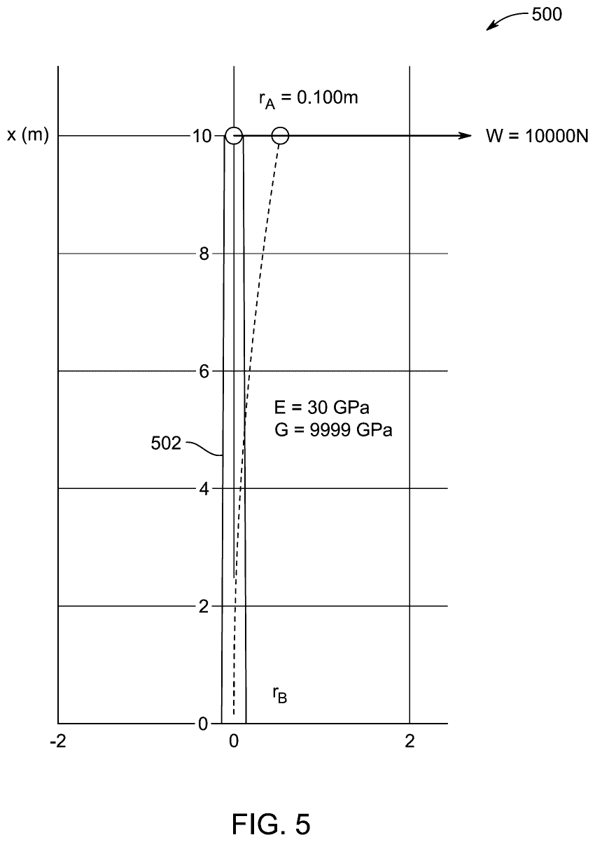

FIG. 5 illustrates an exemplary environment 500 for the computation of the longitudinal MoE (E) and the shearing MoE (G) for a tree trunk 502, according to an exemplary embodiment. The tree trunk 502 is representative of the tree trunks 102, 202, 302, and 402 in FIG. 1, FIG. 2, FIG. 3, and FIG. 4, respectively. In an example, the tree trunk 502 is subjected to a lateral component of force W with a magnitude of 10,000 N at a height of 10 meters from the ground surface. The application of the lateral component of force W causes a predetermined value of lateral deflection y_{A} of the tree trunk 502. The tree trunk 502 has a radius at the tip, r_{A}, of 0.01 m, and a predetermined value for the radius at the base, r_{B}.

Using Equation 23, computed earlier and shown below, and substituting all the described values,

$$E = β \cdot W \cdot {L}^{3} / {y}_{A} \cdot {I}_{A} \qquad ( 23 )$$

E=30 Gpa was found.

Similarly, using Equation 2, 12, 18, and 19, the value of G was computed as 9999 Gpa.

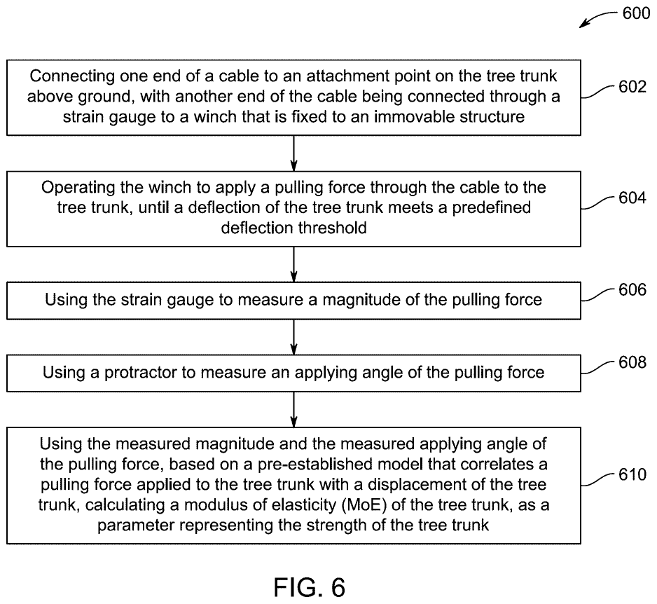

FIG. 6 illustrates a flowchart of method 600 for determining the strength of a tree trunk 102, 202, 302, 402, 502 according to an embodiment. The method 600 is described in conjunction with FIG. 1-FIG. 5. Various steps of the method 600 are included through blocks in FIG. 6. One or more blocks may be combined or eliminated to determine the strength of the tree trunk 102, 202, 302, 402, 502, without departing from the scope of the present disclosure.

At step 602, the method 600 includes connecting one end of a cable 104, 204, 404 to a corresponding attachment point 106, 206, 408 on corresponding the tree trunk 102, 202, 302, 402, 502 above ground 108, 208, 308, with another end of the cable 104, 204, 404 being connected through a strain gauge 110 to a winch 112 that is fixed to an immovable structure 114.

At step 604, the method 600 includes operating the winch 112 to apply a pulling force through the cable 104, 204, 404 to the tree trunk 102, 202, 302, 402, 502, until a deflection of the tree trunk 102, 202, 302, 402, 502 meets a predefined deflection threshold. The predefined deflection threshold refers to the deflection of the point 206, 306, 408 of application of the force F or the point of the deflection of the tip of the tree trunk 102, 202, 302, 402, 502.

At step 606, the method 600 includes using the strain gauge 110 to measure a magnitude of the pulling force F.

At step 608, the method 600 includes using a protractor to measure an applying angle φ of the pulling force F. The angle φ is measured either from the vertical direction or from the horizontal direction. The angle φ may be measured with respect to the location of the winch 112 which the attached cable 104, 204, 404 makes with the tree trunk 102, 202, 302, 402, 502. The measurement of the angle φ is used in the computation of the horizontal component W of the force F.

At step 610, the method 600 includes using the measured magnitude and the measured applying angle φ of the pulling force F, based on a pre-established model that correlates a pulling force F applied to the tree trunk 102, 202, 302, 402, 502 with a displacement of the tree trunk 102, 202, 302, 402, 502, calculating a Modulus of Elasticity (MoE) of the tree trunk 102, 202, 302, 402, 502, as a parameter representing the strength of the tree trunk.

Based upon the aforementioned description of the invention, the disclosure relates to a method for determining the strength of a palm tree trunk through a pull test. One end of a cable is connected to an attachment point on the trunk above the ground, while the other end is connected via a strain gauge to a winch, which is fixed to an immovable structure. The winch is then operated to extend the cable, pulling the trunk until the attachment point or the top of the trunk reaches a predefined horizontal displacement from its rest position. By measuring the pulling force with the strain gauge and measuring the angle of the cable relative to the horizontal or vertical plane, the Modulus of Elasticity (MoE) of the trunk material can be calculated using a model that correlates the pulling force with the trunk displacement. Compared with prior art, this method uses a strain gauge and a protractor instead of sensors or digital equipment, providing a faster and affordable test.

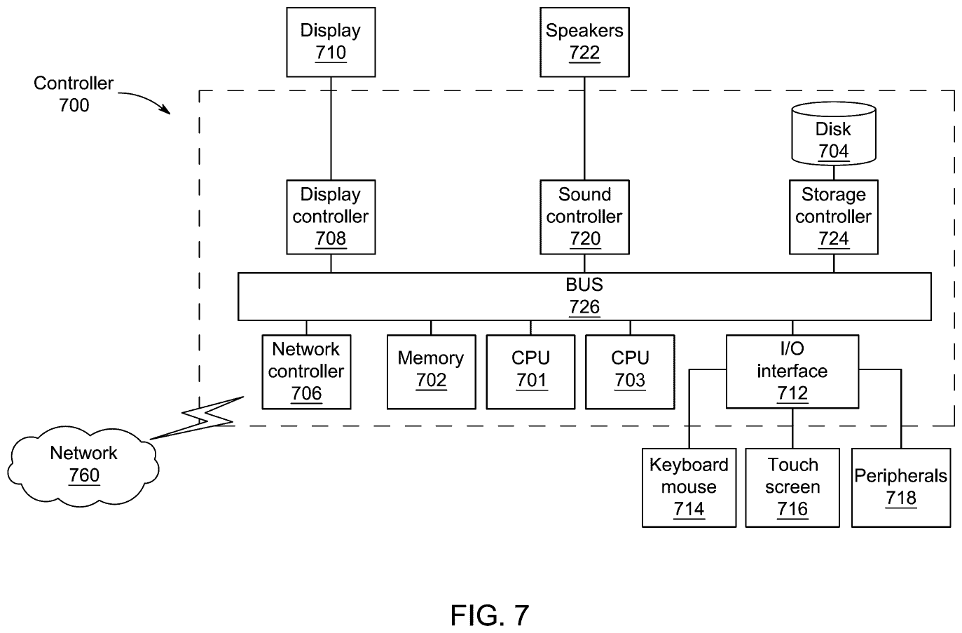

Next, further details of the hardware description of the computing environment according to exemplary embodiments is described with reference to FIG. 7. In FIG. 7, a controller 700 described is representative of the exemplary computer system described in FIG. 1 for automatically controlling the setup for measuring the reading of the strain gauge 110, automatically controlling the winch 112 for applying the pulling force F to the cable 104 and automatically controlling the protractor to measure the angle of inclination of the cable 104 with respect to the ground surface 108 or the vertical plane, in which the controller 700 is a computing device which includes a CPU 701 which performs the processes described above/below. The process data and instructions may be stored in memory 702. These processes and instructions may also be stored on a storage medium disk 704 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 701, 703 and an operating system such as Microsoft Windows 7, Microsoft Windows 10, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, CPU 701 or CPU 703 may be a Xenon or Core processor from Intel of America or an Opteron processor from AMD of America, or may be other processor types that would be recognized by one of ordinary skill in the art. Alternatively, the CPU 701, 703 may be implemented on an FPGA, ASIC, PLD or using discrete logic circuits, as one of ordinary skill in the art would recognize. Further, CPU 701, 703 may be implemented as multiple processors cooperatively working in parallel to perform the instructions of the inventive processes described above.

The computing device in FIG. 7 also includes a network controller 706, such as an Intel Ethernet PRO network interface card from Intel Corporation of America, for interfacing with network 760. As can be appreciated, the network 760 can be a public network, such as the Internet, or a private network such as an LAN or WAN network, or any combination thereof and can also include PSTN or ISDN sub-networks. The network 760 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The computing device further includes a display controller 708, such as a NVIDIA GeForce GTX or Quadro graphics adaptor from NVIDIA Corporation of America for interfacing with display 710, such as a Hewlett Packard HPL2445w LCD monitor. A general purpose I/O interface 712 interfaces with a keyboard and/or mouse 714 as well as a touch screen panel 716 on or separate from display 710. General purpose I/O interface also connects to a variety of peripherals 718 including printers and scanners, such as an OfficeJet or DeskJet from Hewlett Packard.

A sound controller 720 is also provided in the computing device such as Sound Blaster X-Fi Titanium from Creative, to interface with speakers/microphone 722 thereby providing sounds and/or music.

The general-purpose storage controller 724 connects the storage medium disk 704 with communication bus 726, which may be an ISA, EISA, VESA, PCI, or similar, for interconnecting all of the components of the computing device. A description of the general features and functionality of the display 710, keyboard and/or mouse 714, as well as the display controller 708, storage controller 724, network controller 706, sound controller 720, and general purpose I/O interface 712 is omitted herein for brevity as these features are known.

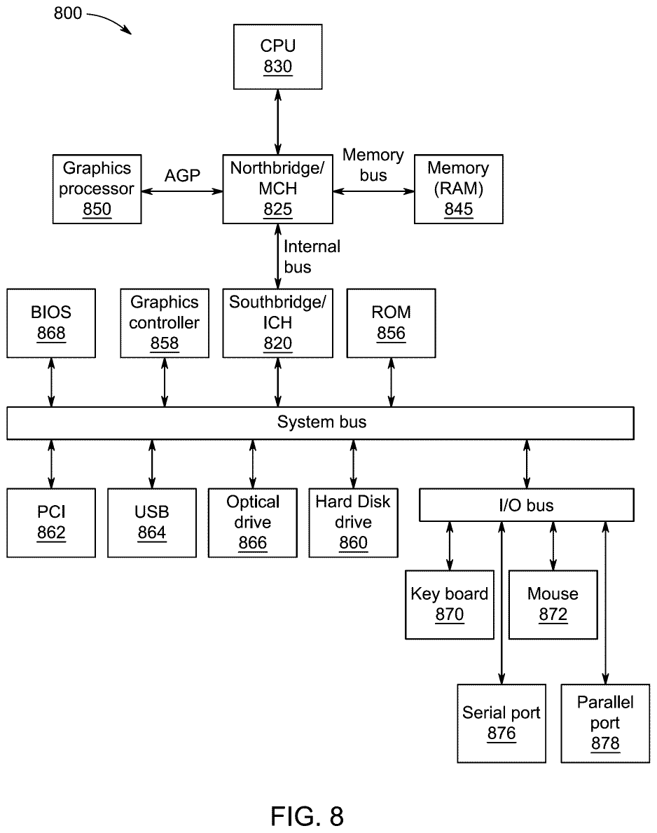

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 8.

FIG. 8 shows a schematic diagram of a data processing system, according to certain embodiments, for performing the functions of the exemplary embodiments. The data processing system is an example of a computer in which code or instructions implementing the processes of the illustrative embodiments may be located.

In FIG. 8, data processing system 800 employs a hub architecture including a north bridge and memory controller hub (NB/MCH) 825 and a south bridge and input/output (I/O) controller hub (SB/ICH) 820. The central processing unit (CPU) 830 is connected to NB/MCH 825. The NB/MCH 825 also connects to the memory 845 via a memory bus, and connects to the graphics processor 850 via an accelerated graphics port (AGP). The NB/MCH 825 also connects to the SB/ICH 820 via an internal bus (e.g., a unified media interface or a direct media interface). The CPU Processing unit 830 may contain one or more processors and even may be implemented using one or more heterogeneous processor systems.

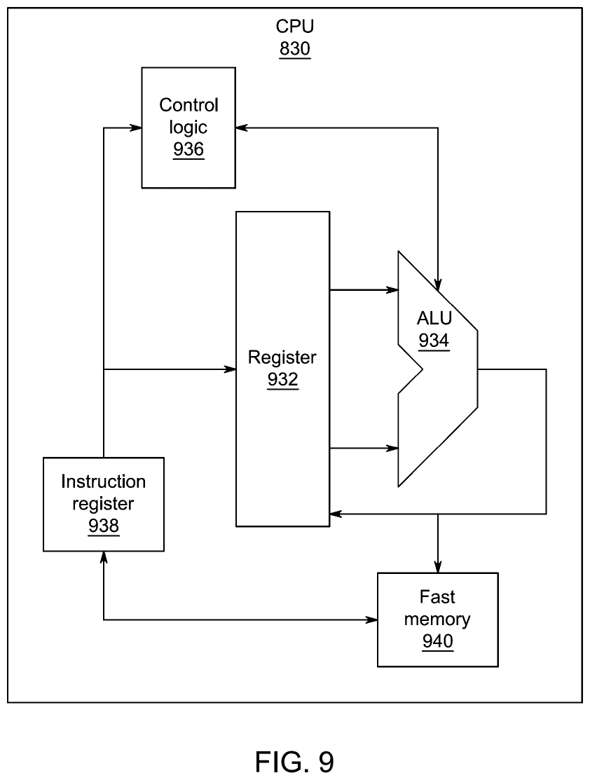

For example, FIG. 9 shows one implementation of CPU 830, according to an embodiment. In one implementation, the instruction register 938 retrieves instructions from the fast memory 940. At least part of these instructions is fetched from the instruction register 938 by the control logic 936 and interpreted according to the instruction set architecture of the CPU 830. Part of the instructions can also be directed to the register 932. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 934 that loads values from the register 932 and performs logical and mathematical operations on the loaded values according to the instructions. The results from these operations can be feedback into the register and/or stored in the fast memory 940. According to certain implementations, the instruction set architecture of the CPU 830 can use a reduced instruction set architecture, a complex instruction set architecture, a vector processor architecture, a very large instruction word architecture. Furthermore, the CPU 830 can be based on the Von Neuman model or the Harvard model. The CPU 830 can be a digital signal processor, an FPGA, an ASIC, a PLA, a PLD, or a CPLD. Further, the CPU 830 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 8, the data processing system 800 can include that the SB/ICH 820 is coupled through a system bus to an I/O Bus, a read only memory (ROM) 856, universal serial bus (USB) port 864, a flash binary input/output system (BIOS) 868, and a graphics controller 858. PCI/PCIe devices can also be coupled to SB/ICH 888 through a PCI bus 862.

The PCI devices may include, for example, Ethernet adapters, add-in cards, and PC cards for notebook computers. The Hard disk drive 860 and CD-ROM 866 can use, for example, an integrated drive electronics (IDE) or serial advanced technology attachment (SATA) interface. In one implementation the I/O bus can include a super I/O (SIO) device.

Further, the hard disk drive (HDD) 860 and optical drive 866 can also be coupled to the SB/ICH 820 through a system bus. In one implementation, a keyboard 870, a mouse 872, a parallel port 878, and a serial port 876 can be connected to the system bus through the I/O bus. Other peripherals and devices that can be connected to the SB/ICH 820 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

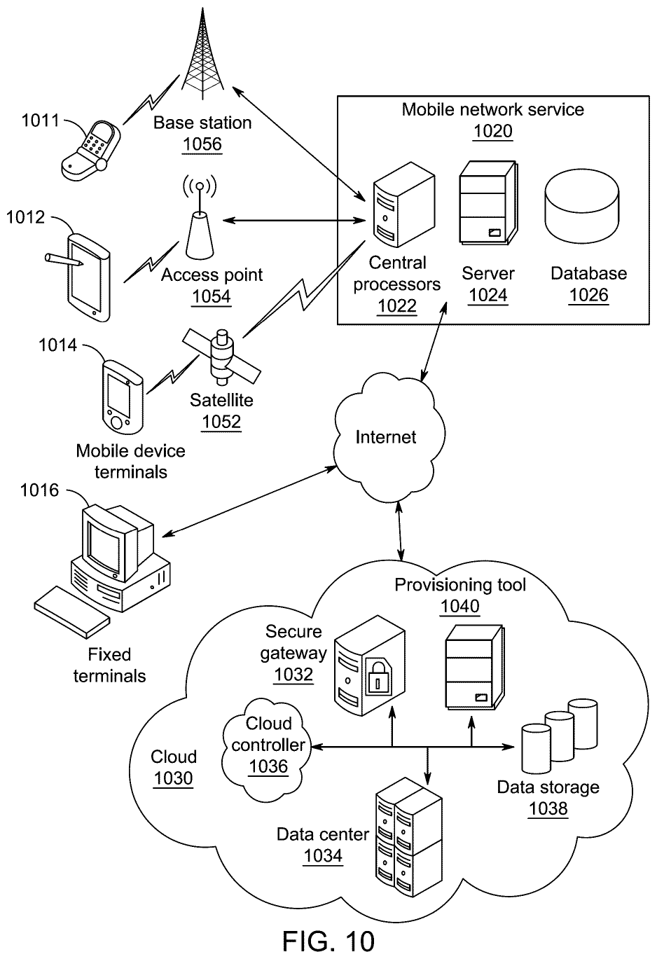

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 10, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A method for determining strength of a tree trunk, comprising:
connecting one end of a cable to an attachment point on the tree trunk above ground, with another end of the cable being connected through a strain gauge to a winch that is fixed to an immovable structure;
operating the winch to apply a pulling force through the cable to the tree trunk, until a deflection of the tree trunk meets a predefined deflection threshold;
using the strain gauge to measure a magnitude of the pulling force;
using a protractor to measure an applying angle of the pulling force; and
using the measured magnitude and the measured applying angle of the pulling force, based on a pre-established model that correlates a pulling force applied to the tree trunk with a displacement of the tree trunk, calculating a Modulus of Elasticity (MoE) of the tree trunk, as a parameter representing the strength of the tree trunk.

2. The method of claim 1, wherein the step of calculating the MoE of the tree trunk further comprises calculating:
a longitudinal MoE along a fiber direction of the tree trunk, and
a shearing MoE along a direction normal to the fiber direction,
as the parameter representing the strength of the tree trunk.

3. The method of claim 1, wherein the pre-established model represents the tree trunk as a tapered solid beam that is clamped at a bottom and free at a tip.

4. The method of claim 3, wherein the pre-established model represents a tree canopy as a mass applied on the tapered solid beam.

5. The method of claim 4, wherein the pre-established model represents the tree canopy as a concentrated mass carried by the tapered solid beam.

6. The method of claim 4, wherein the pre-established model represents the tree canopy as a mass distributed along the tapered solid beam.

7. The method of claim 6, wherein the pre-established model represents the tree canopy as an ellipsoidal shape, with a top of the ellipsoidal shape extending beyond a height of the tapered solid beam.

8. The method of claim 6, wherein the pre-established model represents the tree canopy as an conical shape, with a top of the conical shape extending beyond a height of the tapered solid beam.

9. The method of claim 4, wherein the pre-established model is defined by a plurality of parameters,
values of the plurality of parameters are determined based on geometry and physical properties of the tree trunk and the tree canopy, and
the geometry and physical properties include a length, a cross-section, a tapering of the tree trunk, and a foliage mass of the tree canopy.

10. The method of claim 3, wherein the pre-established model represents the tree trunk as a number (N) of tree trunk elements, and a displacement and a rotation of a i-th tree trunk element are determined based on a shear force and a moment acting at the i-th tree trunk element and a displacement and a rotation of an (i−1)-th tree trunk element.

11. The method of claim 1, wherein the pulling force is applied through the cable to the tree trunk, until the attachment point deflects from a rest position thereof to a predefined distance threshold in a horizontal direction.

12. The method of claim 1, wherein the pulling force is applied through the cable to the tree trunk, until a tip of the tree trunk deflects from a rest position thereof to a predefined distance threshold in a horizontal direction.

13. The method of claim 1, wherein the protractor is used to measure an angle of the cable with respect to a horizontal direction.

14. The method of claim 1, wherein the protractor is used to measure an angle of the cable with respect to a vertical direction.

15. The method of claim 1, wherein the tree trunk has a length of 12 meters, and the pulling force is applied at an applying angle of 45°, until the deflection of the tree trunk reaches 0.5 meter.
